SEQUENCE LISTING (33 sequences)

SEQ ID NO: 1 - DNA, 267 bp, Salmonella enteritidis
ataatgaccagcatgcataagggttcatgccccattgcatagcctgcatgacgatagccatacagtcggctggtttccctgcaaggtgtgccggtaccgtcacctgtgagtctgccatcaggttagcgaaagctgttaactgacccagtgcctgaacgttaaaaattgcgttactggcagaaatggtgtttggtgcctgctgctcagtggtaacaatatttgtgttttccatgattttccccttatgcctgtatgtacggtgtacag

SEQ ID NO: 2 - DNA, 877 bp, Salmonella typhimurium
attatcttttcgccaccataaaaaacttggagaaaaaatagcattaaaagcaggcatgtccaccggtgacaatattaaatttcaaagatattggtacgaggtttcaataaaaaaaacccttatcacaaataaagaatcaaatacaaaaatcgacattcataatatcaaatggtttccttgtagtagtggaggtgaatatcgaaagtggtatggtaataacgaaatagttgtaaattgggaaaataatggttacgaaatacgaaattttaaatttgagaatggcaaaactcgctctgccgtaagaaatgatgagtattactttagagaaggcataacatggtcaaaaataagccaaggtaatttttgcgtgagatatcgtccaaaagggtttgtttttgatgatacaggccgttgcggcttttcaaataacaaaaatgagttgctttacgctgcgggattaatgtgcactccggttgtaaaccattatttatcaatactagcccccacacttagctttactagtggtgaattagcctcagtaccatatccagaaattgaagatgaaattatcgaattagtcaccaatgctattgaaatagctaaaaatgactgggactctcaagagcaatcatgggattatgtttgttcaccattgcttgaacacaattcaactcaattgctacggaatatctacaaacaaaaaatcaatacaaatatcaagttagtcgaaacacttctcctaatagaaaatactataaataatatttttatagataaattacaacttgacaaaactataattaaagctgtattgcaaagtgaaataactctactatgtaatccaaactatcgatataaaaatattcaagatcatacc

SEQ ID NO: 3 - DNA, 25 bp, Artificial Sequence
  Primer
ataatgaccagcatgcataagggtt

SEQ ID NO: 4 - DNA, 27 bp, Artificial Sequence
  Primer
ctgtacaccgtacatacaggcataagg

SEQ ID NO: 5 - DNA, 30 bp, Artificial Sequence
  Probe
actgtatggctatcgtcatgcaggctatgc

SEQ ID NO: 6 - DNA, 30 bp, Artificial Sequence
  Quencher
gcatagcctgcatgacgatagccatacagt

SEQ ID NO: 7 - DNA, 26 bp, Artificial Sequence
  Primer
tgcgtgagatatcgtccaaaagggtt

SEQ ID NO: 8 - DNA, 34 bp, Artificial Sequence
  Primer
gctaagtgtgggggctagtattgataaataatgg

SEQ ID NO: 9 - DNA, 33 bp, Artificial Sequence
  Primer
tttcaaataacaaaaatgagttgctttacgctg

SEQ ID NO: 10 - DNA, 33 bp, Artificial Sequence
  Primer
agcattggtgactaattcgataatttcatcttc

SEQ ID NO: 11 - DNA, 27 bp, Artificial Sequence
  Primer
caaataacaaaaatgagttgctttacg

SEQ ID NO: 12 - DNA, 30 bp, Artificial Sequence
  Primer
gcattggtgactaattcgataatttcatct

SEQ ID NO: 13 - DNA, 27 bp, Artificial Sequence
  Primer
aatggtttccttgtagtagtggaggtg

SEQ ID NO: 14 - DNA, 26 bp, Artificial Sequence
  Primer
catcatttcttacggcagagcgagtt

SEQ ID NO: 15 - DNA, 32 bp, Artificial Sequence
  Probe
taatggtttacaaccggagtgcacattaatcc

SEQ ID NO: 16 - DNA, 26 bp, Artificial Sequence
  Quencher
atgtgcactccggttgtaaaccatta

SEQ ID NO: 17 - DNA, 34 bp, Artificial Sequence
  Probe
tgataaataatggtttacaaccggagtgcacatt

SEQ ID NO: 18 - DNA, 30 bp, Artificial Sequence
  Quencher
tgcactccggttgtaaaccattatttatca

SEQ ID NO: 19 - DNA, 34 bp, Artificial Sequence
  Probe
agtggtgaattagcctcagtaccatatccagaaa

SEQ ID NO: 20 - DNA, 29 bp, Artificial Sequence
  Quencher
ggatatggtactgaggctaattcaccact

SEQ ID NO: 21 - DNA, 31 bp, Artificial Sequence
  Probe
cgctgcgggattaatgtgcactccggttgta

SEQ ID NO: 22 - DNA, 31 bp, Artificial Sequence
  Quencher
tacaaccggagtgcacattaatcccgcagcg

SEQ ID NO: 23 - DNA, 25 bp, Artificial Sequence
  Quencher
cggagtgcacattaatcccgcagcg

SEQ ID NO: 24 - DNA, 34 bp, Artificial Sequence; identical to SEQ ID NO: 19
  Probe

SEQ ID NO: 25 - DNA, 34 bp, Artificial Sequence
  Quencher
tttctggatatggtactgaggctaattcaccact

SEQ ID NO: 26 - DNA, 29 bp, Artificial Sequence
  Quencher
ggatatggtactgaggctaattcaccact

SEQ ID NO: 27 - DNA, 146 bp, Simian virus 40
gagcatagttattaatagcagacactctatgcctgtgtggagtagcggcggcggtcgtctgtataaaggttacagaatatttttccataattttcttgtatagcagtgcagctttttcctttgtggtgtaaatagcaaagcaagca

SEQ ID NO: 28 - DNA, 27 bp, Artificial Sequence
  Primer
accagctgataatgaccagcatgcata

SEQ ID NO: 29 - DNA, 27 bp, Artificial Sequence
  Primer
cgttccctgtacaccgtacatacaggc

SEQ ID NO: 30 - DNA, 27 bp, Artificial Sequence
  Primer
tagcaccagctgataatgaccagcatg

SEQ ID NO: 31 - DNA, 27 bp, Artificial Sequence
  Primer
gtcaggcgttccctgtacaccgtacat

SEQ ID NO: 32 - DNA, 24 bp, Artificial Sequence
  Primer
cgtgagatatcgtccaaaagggtt

SEQ ID NO: 33 - DNA, 29 bp, Artificial Sequence
  Primer
taagtgtgggggctagtattgataaataa

(19) United States
(12) Patent Application Publication
(10) Pub. No.: US 2016/0201116 A1
(43) Pub. Date: Jul. 14, 2016

(54) SEQUENCES AND THEIR USE FOR DETECTION OF SALMONELLA ENTERITIDIS AND/OR SALMONELLA TYPHIMURIUM

(71) Applicant: E. I. DU PONT DE NEMOURS AND COMPANY, Wilmington, DE (US)

(72) Inventors: Mark A. Jensen, West Chester, PA (US); Daniel R. Demarco, Wilmington, DE (US); Stephen Varkey, Newark, DE (US); Jun Li, Chadds Ford, PA (US)

(21) Appl. No.: 14/913,212

(22) PCT Filed: Aug. 19, 2014
(86) PCT No.: PCT/US2014/051575

§ 371 Date: Feb. 19, 2016

Related U.S. Application Data
(60) Provisional application No. 61/867,754, filed on Aug. 20, 2013.

(51) Int. Cl. C12Q 1/68

(52) U.S. Cl. CPC C12Q 1/689; C12Q 2600/16

(57) ABSTRACT

This invention relates to a rapid, accurate method for detection and characterization of Salmonella enteritidis and/or Salmonella typhimurium based on the presence of nucleic acid sequences, in particular, to a PCR-based method for detection, and to oligonucleotide molecules and reagents and kits useful therefore. This method is preferably employed to detect S. enteritidis and/or S. typhimurium in an environmental sample. The present invention further relates to replication compositions and kits for carrying out the method of the present invention.

### CROSS REFERENCE TO RELATED APPLICATIONS

This application claims priority of U.S. Provisional Application Ser. No. 61/867,754 filed Aug. 20, 2013, which is hereby incorporated by reference in its entirety.

### FIELD OF INVENTION

The field of invention relates to methods for detection and characterization of Salmonella enteritidis and/or Salmonella typhimurium based on the presence of nucleic acid sequences, preferably PCR-based methods for detection, and to oligonucleotide molecules and reagents and kits useful therefor.

### BACKGROUND OF INVENTION

Salmonella enteritidis and Salmonella typhimurium are the predominant serovars of Salmonella associated with human disease in most countries. S. enteritidis and S. typhimurium are noted separately from other Salmonella serovars for two reasons: (1) these two serovars are often specifically cited in zoonosis control legislation and (2) differences in the epidemiology as compared to other salmonellae.

S. enteritidis, especially phage type 4, has become much more common in both poultry and humans since the early 80s. The prevalence of S. typhimurium has remained relatively stable, though the spread of the highly antibiotic-resistant strain DT104 in various farmed species gives some reason for concern. Infections in chickens, turkeys and ducks cause problems worldwide with morbidity of 0-90% and a low to moderate mortality. Many infected birds are culled and others are rejected at slaughter. The route of infection is oral; many species are intestinal carriers and infection may be carried by faeces, fomites and on eggshells.

Currently, Salmonella isolates are typed using the White-Kauffmann-Le Minor scheme. This classification scheme is utilized by public health organizations worldwide and is considered the standard for the determination of Salmonella serotypes. The White-Kauffmann-Le Minor scheme subtypes Salmonella into serotypes on the basis of surface antigen identification using polyclonal antiserum to determine the O (somatic) and H (flagellar) antigenic epitopes. Serotyping is essential for human disease surveillance and outbreak detection, as both the virulence and host range of Salmonella isolates can be serotype specific. Despite its usefulness, traditional serotyping is labor-intensive and expensive and can take up to five days to complete. It requires specialized expertise and a set of more than 250 stringently quality-assured reagents to characterize the more than 2,500 Salmonella serovars. Many hospital and private laboratories rely on the use of a limited number of commercially available antisera, covering only a restricted number of serotypes. These laboratories are forced to ship isolates to reference laboratories for full serotyping, causing delays in isolate identification that ultimately impede progress in outbreak investigations and containment.

Therefore, there is a need for assays that detect S. enteritidis and/or S. typhimurium with fast time-to results, high accuracy, and superior ease of use.

### SUMMARY OF INVENTION

One aspect is for a method for detecting the presence of Salmonella enteritidis and/or Salmonella typhimurium in a sample, said sample comprising nucleic acids, said method comprising: (a) providing a reaction mixture comprising suitable primer pairs for amplification of at least a portion of (i) a Salmonella enteritidis SEN0908A/SEN0909/SEN0910 region, and/or (ii) a Salmonella typhimurium type II restriction enzyme methylase region; (b) performing PCR amplification of said nucleic acids of said sample using the reaction mixture of step (a); and (c) detecting the amplification of step (b), whereby a positive detection of amplification indicates the presence of Salmonella enteritidis and/or Salmonella typhimurium in the sample.

Another aspect is for a primer comprising a polynucleotide sequence having at least 95% sequence identity based on the BLASTN method of alignment to the polynucleotide sequence set forth in SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:28, SEQ ID NO:29, SEQ ID NO:30, SEQ ID NO:31, SEQ ID NO:32, or SEQ ID NO:33.

A further aspect is for a probe/quencher pair comprising polynucleotide sequences having at least 95% sequence identity based on the BLASTN method of alignment to the polynucleotide sequences set forth in SEQ ID NO:5 and SEQ ID NO:6, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, or SEQ ID NO:24 and SEQ ID NO:26.

An additional aspect is for a Salmonella enteritidis or Salmonella typhimurium detection sequence comprising a polynucleotide sequence having at least 95% sequence identity based on the BLASTN method of alignment to the polynucleotide sequence set forth in SEQ ID NO:1 or SEQ ID NO:2.

A further aspect is for an isolated polynucleotide comprising a polynucleotide sequence having at least 95% sequence identity based on the BLASTN method of alignment to the polynucleotide sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:28, SEQ ID NO:29, SEQ ID NO:30, SEQ ID NO:31, SEQ ID NO:32, or SEQ ID NO:33.

Another aspect is for replication composition for use in performance of PCR, comprising: (a) a set of primer pairs selected from the group consisting of: (i) one or more primer pairs comprising nucleic acid sequences comprising: (A) SEQ ID NO:3 and SEQ ID NO:4; (B) SEQ ID NO:28 and SEQ ID NO:29; and/or (C) SEQ ID NO:30 and 31; (ii) one or more primer pairs comprising nucleic acid sequences comprising: (A) SEQ ID NO:7 and SEQ ID NO:8; (B) SEQ ID NO:9 and SEQ ID NO:10; (C) SEQ ID NO:11 and SEQ ID NO:12; (D) SEQ ID NO:13 and SEQ ID NO:14; and/or (E) SEQ ID NO:32 and SEQ ID NO:33; and (iii) a combination thereof; and (b) at least one thermostable DNA polymerase.

An additional aspect is for a kit for detection of Salmonella enteritidis in a sample, comprising the aforementioned replication composition, wherein the kit comprises the primer pair of (a)(i) and the probe/quencher pair of SEQ ID NO:5 and SEQ ID NO:6.

A further aspect is for a kit for detection of Salmonella typhimurium in a sample, comprising the aforementioned replication composition, wherein the kit comprises the one or more primer pairs of (a)(ii) and at least one probe/quencher pair selected from the group consisting of SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, or SEQ ID NO:24 and SEQ ID NO:26, and a combination thereof.

Another aspect is for a kit for the detection of Salmonella enteritidis and/or Salmonella typhimurium in a sample, comprising the aforementioned replication composition, wherein the kit comprises the primer pair of (a)(i); the one or more primer pairs of (a)(ii); the probe/quencher pair of SEQ ID NO:5 and SEQ ID NO:6; and at least one probe/quencher pair selected from the group consisting of SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, or SEQ ID NO:24 and SEQ ID NO:26, and a combination thereof.

Other objects and advantages will become apparent to those skilled in the art upon reference to the detailed description that hereinafter follows.

### SUMMARY OF THE SEQUENCES

The MD6691 Sequence Listing is attached as Appendix A and incorporated herein.

The sequences conform with 37 C.F.R. §§1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NO:1 is a Salmonella enteritidis SEN0908A/SEN0909/SEN0910 region sequence for detection.

SEQ ID NO:2 is a Salmonella typhimurium type II restriction enzyme methylase region for detection.

SEQ ID NO:3 is a forward primer for detection of S. enteritidis.

SEQ ID NO:4 is a reverse primer for detection of S. enteritidis.

SEQ ID NO:5 is a probe for use in the detection of S. enteritidis. In one embodiment, the probe is 5′-labeled with a fluorescent dye. In some embodiments, the 3′ terminus of SEQ ID NO:5 is attached to the 5′ terminus of one of the primers listed above, preferably SEQ ID NO:3, via a suitable linker moiety, such as a hexethylene glycol (HEG) spacer consisting of 6 ethylene glycol units.

SEQ ID NO:6 is a blocking oligonucleotide (quencher) capable of hybridizing to the probe of SEQ ID NO:5. In one embodiment, this blocking oligonucleotide is 3′-labeled with a fluorescent dye.

SEQ ID NO:7 is a forward primer for detection of S. typhimurium.

SEQ ID NO:8 is a reverse primer for detection of S. typhimurium.

SEQ ID NO:9 is a forward primer for detection of S. typhimurium.

SEQ ID NO:10 is a reverse primer for detection of S. typhimurium.

SEQ ID NO:11 is a forward primer for detection of S. typhimurium.

SEQ ID NO:12 is a reverse primer for detection of S. typhimurium.

SEQ ID NO:13 is a forward primer for detection of S. typhimurium.

SEQ ID NO:14 is a reverse primer for detection of S. typhimurium.

SEQ ID NO:15 is a probe for use in the detection of S. typhimurium. In one embodiment, the probe is 5′-labeled with a fluorescent dye. In some embodiments, the 3′ terminus of SEQ ID NO:15 is attached to the 5′ terminus of one of the primers listed above, preferably SEQ ID NO: 9 or 11 (or a fragment thereof), via a suitable linker moiety, such as a hexethylene glycol spacer consisting of 6 ethylene glycol units.

SEQ ID NO:16 is a blocking oligonucleotide (quencher) capable of hybridizing to the probe of SEQ ID NO:15. In one embodiment, this blocking oligonucleotide is 3′-labeled with a fluorescent dye.

SEQ ID NO:17 is a probe for use in the detection of S. typhimurium. In one embodiment, the probe is 5′-labeled with a fluorescent dye. In some embodiments, the 3′ terminus of SEQ ID NO:17 is attached to the 5′ terminus of one of the primers listed above, preferably SEQ ID NO: 9 or 11 (or a fragment thereof), via a suitable linker moiety, such as a hexethylene glycol spacer consisting of 6 ethylene glycol units.

SEQ ID NO:18 is a blocking oligonucleotide (quencher) capable of hybridizing to the probe of SEQ ID NO:17. In one embodiment, this blocking oligonucleotide is 3′-labeled with a fluorescent dye.

SEQ ID NO:19 is a probe for use in the detection of S. typhimurium. In one embodiment, the probe is 5′-labeled with a fluorescent dye. In some embodiments, the 3′ terminus of SEQ ID NO:19 is attached to the 5′ terminus of one of the primers listed above, preferably SEQ ID NO:12 (or a fragment thereof), via a suitable linker moiety, such as a hexethylene glycol spacer consisting of 6 ethylene glycol units.

SEQ ID NO:20 is a blocking oligonucleotide (quencher) capable of hybridizing to the probe of SEQ ID NO:19. In one embodiment, this blocking oligonucleotide is 3′-labeled with a fluorescent dye.

SEQ ID NO:21 is a probe for use in the detection of S. typhimurium. In one embodiment, the probe is 5′-labeled with a fluorescent dye. In some embodiments, the 3′ terminus of SEQ ID NO:21 is attached to the 5′ terminus of one of the primers listed above, preferably SEQ ID NO:8 (or a fragment thereof), via a suitable linker moiety, such as a hexethylene glycol spacer consisting of 6 ethylene glycol units.

SEQ ID NO:22 is a blocking oligonucleotide (quencher) capable of hybridizing to the probe of SEQ ID NO:21. In one embodiment, this blocking oligonucleotide is 3′-labeled with a fluorescent dye.

SEQ ID NO:23 is a blocking oligonucleotide (quencher) capable of hybridizing to the probe of SEQ ID NO:21. In one embodiment, this blocking oligonucleotide is 3′-labeled with a fluorescent dye.

SEQ ID NO:24 is a probe for use in the detection of S. typhimurium. In one embodiment, the probe is 5′-labeled with a fluorescent dye. In some embodiments, the 3′ terminus of SEQ ID NO:24 is attached to the 5′ terminus of one of the primers listed above, preferably SEQ ID NO:10 (or a fragment thereof), via a suitable linker moiety, such as a hexethylene glycol spacer consisting of 6 ethylene glycol units.

SEQ ID NO:25 is a blocking oligonucleotide (quencher) capable of hybridizing to the probe of SEQ ID NO:24. In one embodiment, this blocking oligonucleotide is 3′-labeled with a fluorescent dye.

SEQ ID NO:26 is a blocking oligonucleotide (quencher) capable of hybridizing to the probe of SEQ ID NO:24. In one embodiment, this blocking oligonucleotide is 3′-labeled with a fluorescent dye.

SEQ ID NO:27 is a PCR control sequence from Simian Virus 40 (SV40).

SEQ ID NO:28 is a forward primer for detection of S. enteritidis.

SEQ ID NO:29 is a reverse primer for detection of S. enteritidis.

SEQ ID NO:30 is a forward primer for detection of S. enteritidis.

SEQ ID NO:31 is a reverse primer for detection of S. enteritidis.

SEQ ID NO:32 is a forward primer for detection of S. typhimurium.

SEQ ID NO:33 is a reverse primer for detection of S. typhimurium.

### DETAILED DESCRIPTION OF PREFERRED EMBODIMENTS

Applicants specifically incorporate the entire contents of all cited references in this disclosure. Further, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

### DEFINITIONS

In this disclosure, a number of terms and abbreviations are used. The following definitions are provided.

As used herein, the term “about” or “approximately” means within 20%, preferably within 10%, and more preferably within 5% of a given value or range.

The term “comprising” is intended to include embodiments encompassed by the terms “consisting essentially of” and “consisting of”. Similarly, the term “consisting essentially of” is intended to include embodiments encompassed by the term “consisting of”.

The term “Salmonella enteritidis SEN0908A/SEN0909/SEN0910 region” refers to the area of the S. enteritidis genome that is at the junctions of three hypothetical proteins. In the Salmonella enterica subsp. enterica serovar Enteritidis str. P125109 chromosome complete genome (NCBI Reference Sequence: NC_011294.1), these three hypothetical proteins have the following locus tags: SEN0908A, SEN0909, and SEN0910. In some embodiments, the target region of S. enteritidis is SEQ ID NO:1 and is located between positions 1013560 and 1013826 in the chromosome of S. enteritidis strain P125109. SEQ ID NO:1 has 267 nucleotides and 45.3% G+C content.

The term “Salmonella typhimurium type II restriction enzyme methylase region” refers to the area of the S. typhimurium genome that encodes a type II restriction enzyme methylase. In some embodiments, the target region of S. typhimurium is SEQ ID NO:2 and is located between positions 4766053 and 4766929 in the chromosome of S. typhimurium strain D23580. SEQ ID NO:2 has 877 nucleotides and 32.3% G+C content.

“Polymerase chain reaction” is abbreviated PCR.

The term “isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

The terms “polynucleotide”, “polynucleotide sequence”, “nucleic acid sequence”, and “nucleic acid fragment” are used interchangeably herein. These terms encompass nucleotide sequences and the like. A polynucleotide may be a polymer of RNA or DNA that is single- or double-stranded, that optionally contains synthetic, non-natural, or altered nucleotide bases. A polynucleotide in the form of a polymer of DNA may be comprised of one or more strands of cDNA, genomic DNA, synthetic DNA, or mixtures thereof.

The term “amplification product” refers to nucleic acid fragments produced during a primer-directed amplification reaction. Typical methods of primer-directed amplification include polymerase chain reaction (PCR), ligase chain reaction (LCR), or strand displacement amplification (SDA). If PCR methodology is selected, the replication composition may comprise the components for nucleic acid replication, for example: nucleotide triphosphates, two (or more) primers with appropriate sequences, thermostable polymerase, buffers, solutes, and proteins. These reagents and details describing procedures for their use in amplifying nucleic acids are provided in U.S. Pat. Nos. 4,683,202 and 4,683,195, incorporated herein by reference. If LCR methodology is selected, then the nucleic acid replication compositions may comprise, for example: a thermostable ligase (e.g., Thermus aquaticus ligase), two sets of adjacent oligonucleotides (wherein one member of each set is complementary to each of the target strands), Tris-HCl buffer, KCl, EDTA, NAD, dithiothreitol, and salmon sperm DNA. See, e.g., Tabor et al., Proc. Natl. Acad. Sci. U.S.A. 82:1074-78 (1985).

The term “primer” refers to an oligonucleotide (synthetic or occurring naturally) that is capable of acting as a point of initiation of nucleic acid synthesis or replication along a complementary strand when placed under conditions in which synthesis of a complementary strand is catalyzed by a polymerase. A primer can further contain a detectable label, for example a 5′ end label.

The term “probe” refers to an oligonucleotide (synthetic or occurring naturally) that is complementary (though not necessarily fully complementary) to a polynucleotide of interest and forms a duplexed structure by hybridization with at least one strand of the polynucleotide of interest. A probe or primer-probe complex can further contain a detectable label.

A probe can either be an independent entity or complexed with or otherwise attached to a primer, such as where a probe is connected via its 3′ terminus to a primer's 5′ terminus through a linker, which may be a nucleotide or non-nucleotide linker and which may be a non-amplifiable linker, such as hexethylene glycol (HEG). In such a case, this would be termed a “primer-probe complex”. One example of such a primer-probe complex can be found in U.S. Pat. No. 6,326,145, incorporated herein by reference in its entirety, which are frequently referred to as “Scorpion probes” or “Scorpion primers”.

As used herein, the terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, semiconductor nanocrystals, ligands (e.g., biotin, avidin, streptavidin, or haptens), and the like. A detectable label can also include a combination of a reporter and a quencher.

The term “reporter” refers to a substance or a portion thereof which is capable of exhibiting a detectable signal, which signal can be suppressed by a quencher. The detectable signal of the reporter is, e.g., fluorescence in the detectable range. The term “quencher” refers to a substance or portion thereof which is capable of suppressing, reducing, inhibiting, etc., the detectable signal produced by the reporter.

As used herein, the terms “quenching” and “fluorescence energy transfer” refer to the process whereby, when a reporter and a quencher are in close proximity, and the reporter is excited by an energy source, a substantial portion of the energy of the excited state nonradiatively transfers to the quencher where it either dissipates nonradiatively or is emitted at a different emission wavelength than that of the reporter.

Preferably, the reporter may be selected from fluorescent organic dyes modified with a suitable linking group for attachment to the oligonucleotide, such as to the terminal 3′ carbon or terminal 5′ carbon. The quencher may also be selected from organic dyes, which may or may not be fluorescent, depending on the embodiment of the present invention. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules, 2nd ed., Academic Press, New York, 1971, the content of which is incorporated herein by reference. Examples of modifying reporters and quenchers for covalent attachment via common reactive groups that can be added to an oligonucleotide in the present invention may be found, for example, in Haugland, Handbook of Fluorescent Probes and Research Chemicals, Molecular Probes of Eugene, Oreg., 1992, the content of which is incorporated herein by reference.

Preferred reporter-quencher pairs may be selected from xanthene dyes including fluoresceins and rhodamine dyes. Many suitable forms of these compounds are available commercially with substituents on the phenyl groups, which can be used as the site for bonding or as the bonding functionality for attachment to an oligonucleotide. Another preferred group of fluorescent compounds for use as reporters are the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like.

Most preferably, the reporters and quenchers are selected from fluorescein and rhodamine dyes. These dyes and appropriate linking methodologies for attachment to oligonucleotides are well known in the art.

Suitable examples of quenchers may be selected from 6-carboxy-tetramethyl-rhodamine, 4-(4-dimethylaminophenylazo) benzoic acid (DABYL), tetramethylrhodamine (TAMRA), BHQ-0™, BHQ-1™ BHQ-2™, and BHQ-3™, each of which are available from Biosearch Technologies, Inc. of Novato, Calif., QSY-7™ QSY-9™, QSY-21™ and QSY-35™, each of which are available from Molecular Probes, Inc., and the like.

Suitable examples of reporters may be selected from dyes such as SYBR green, 5-carboxyfluorescein (5-FAM™ available from Applied Biosystems of Foster City, Calif.), 6-carboxyfluorescein (6-FAM), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from Applied Biosystems, Cal Fluor® dye products (such as, e.g., Cal Fluor® Gold 540, Orange 560, Red 590, Red 610, Red 635) available from Biosearch Technologies, Quasar dye products (such as, e.g., Quasar 570, 670, 705) available from Biosearch Technologies, and the like.

One example of a probe which contains a reporter and a quencher is a Scorpion probe in either a unimolecular or bimolecular conformation. In a unimolecular Scorpion, the probe portion of the primer-probe complex is flanked by self-complementary regions which allow the probe to form into a stem-loop structure when the probe is unbound from its target DNA. Examples of such self-complementary regions can be found in SEQ ID NO:5 and SEQ ID NO:6, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, and SEQ ID NO:24 and SEQ ID NO:26. Further, in a unimolecular Scorpion, a reporter is typically attached at or near one of the self-complementary regions, such as at the 5′ terminus of the Scorpion probe, and a quencher is attached at or near the other self-complementary region, such as immediately 5′ to the non-amplifiable linker, such that the quencher is in sufficiently close proximity to the reporter to cause quenching when the probe is in its stem-loop conformation. In a bimolecular Scorpion, self-complementary flanking regions are not typically employed, but rather a separate “blocking oligonucleotide” is employed in conjunction with the Scorpion probe. This blocking oligonucleotide is capable of hybridizing to the probe region of the Scorpion probe when the probe is unbound from its target DNA. An example of a bimolecular Scorpion pair is SEQ ID NO:5 (the Scorpion probe) and SEQ ID NO:6 (the blocking oligonucleotide). Further, in a bimolecular Scorpion, the reporter is typically attached to the probe region of the Scorpion probe, such as at the 5′ terminus of the Scorpion probe, while the quencher is attached to the blocking oligonucleotide, such as at the 3′ terminus of the blocking oligonucleotide, such that the quencher is in sufficiently close proximity to the reporter to cause quenching when the probe is unbound from its target DNA and is instead hybridized to the blocking oligonucleotide.

Another example of a probe which contains a reporter and a quencher is a probe that is to be used in a 5′-exonuclease assay, such as the Taqman® real-time PCR technique. In this context, the oligonucleotide probe will have a sufficient number of phosphodiester linkages adjacent to its 5′ end so that the 5′ to 3′ nuclease activity employed can efficiently degrade the bound probe to separate the reporters and quenchers. Yet another example of a probe which contains a reporter and quencher is a Molecular Beacon type probe, which contains a probe region flanked by self-complementary regions that allow the probe to form a stem-loop structure when unbound from the probe's target sequence. Such probes typically have a reporter attached at or near one terminus and a quencher attached at or near the other terminus such that the quencher is in sufficiently close proximity to the reporter to cause quenching when the probe is in its unbound, and thus stem-loop, form.

The term “replication inhibitor moiety” refers to any atom, molecule or chemical group that is attached to the 3′ terminal hydroxyl group of an oligonucleotide that will block the initiation of chain extension for replication of a nucleic acid strand. Examples include, but are not limited to: 3′-deoxynucleotides (e.g., cordycepin), dideoxynucleotides, phosphate, ligands (e.g., biotin and dinitrophenol), reporter molecules (e.g., fluorescein and rhodamine), carbon chains (e.g., propanol), a mismatched nucleotide or polynucleotide, or peptide nucleic acid units. The term “non-participatory” refers to the lack of participation of a probe or primer in a reaction for the amplification of a nucleic acid molecule. Specifically a non-participatory probe or primer is one that will not serve as a substrate for, or be extended by, a DNA or RNA polymerase. A “non-participatory probe” is inherently incapable of being chain extended by a polymerase. It may or may not have a replication inhibitor moiety.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. Hybridization and washing conditions are well known and exemplified, for example, in Sambrook, J., Fritsch, E. F. and Maniatis, T., Molecular Cloning: A Laboratory Manual, 2nd ed., Cold Spring Harbor Laboratory: Cold Spring Harbor, N.Y. (1989), particularly Chapter 11 and Table 11.1 therein (entirely incorporated herein by reference). The conditions of temperature and ionic strength determine the “stringency” of the hybridization. For preliminary screening for homologous nucleic acids, low stringency hybridization conditions, corresponding to a Tm of 55° C., can be used, e.g., 5×SSC, 0.1% SDS, 0.25% milk, and no formamide; or 30% formamide, 5×SSC, 0.5% SDS. Moderate stringency hybridization conditions correspond to a higher Tm, e.g., 40% formamide, with 5× or 6×SSC. Hybridization requires that the two nucleic acids contain complementary sequences, although, depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one preferred embodiment, the length for a hybridizable nucleic acid is at least about 10 nucleotides. More preferably a minimum length for a hybridizable nucleic acid is at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, or, most preferably, at least 30 nucleotides. Furthermore, the skilled artisan will recognize that the temperature and wash solution salt concentration may be adjusted as necessary according to factors such as length of the probe.

Standard recombinant DNA and molecular cloning techniques used here are well known in the art and are described by, e.g., Sambrook et al. (supra); and by Ausubel, F. M. et al., Current Protocols in Molecular Biology, published by Greene Publishing Assoc. and Wiley-Interscience (1987).

### Genome Detection Regions

Applicants have solved the stated problem through a method that uses a S. enteritidis and/or S. typhimurium detection assay developed based on identification of the Salmonella enteritidis SEN0908A/SEN0909/SEN0910 region and/or the Salmonella typhimurium type II restriction enzyme methylase region. In some embodiments, the assay incorporates unlabeled primers and Scorpion probes for detection of S. enteritidis and/or S. typhimurium (and, in some embodiments, an internal positive control (e.g., sSV40)). In some embodiments, the assay also contains a passive reference for fluorescence signal normalization and offset well-to-well signal variation.

The presently disclosed detection assay has an analytical sensitivity at 10⁴ cfu/mL or lower with both S. enteritidis and S. typhimurium in liquid cultures. Inclusivity testing with approximately 30 strains of S. enteritidis and 50 strains of S. typhimurium showed that all were detected at comparable cell densities. Exclusivity studies with approximately 30 strains of closely-related non-Salmonella species and >100 strains of other Salmonella serovars showed no cross reaction. Testing using food enrichment samples revealed excellent sensitivity and specificity, and robustness to PCR inhibitors.

The present disclosure therefore relates to detection and identification of S. enteritidis and/or S. typhimurium through the detection of the Salmonella enteritidis SEN0908A/SEN0909/SEN0910 region and/or the Salmonella typhimurium type II restriction enzyme methylase region. The present detection method finds utility in detection of S. enteritidis and/or S. typhimurium in any type of sample, for example in appropriate samples for food testing, environmental testing, or human or animal diagnostic testing. While examples of suitable methods for detecting these regions are included herein, it is to be understood that the invention is not limited to the methods described. Rather any suitable method can be employed to detect these DNA regions and subsequently S. enteritidis and/or S. typhimurium in a sample.

### Oligonucleotides

Oligonucleotides of the instant invention are set forth in SEQ ID NOs: 3-26.

Oligonucleotides of the instant invention may be used as primers for PCR amplification. Preferred primer pairs and their corresponding targets, blocking oligonucleotides, and probes are shown in Table 1.

TABLE 1

| 5′ (Forward) | Blocking | 3′ (Reverse) | |
|---|---|---|---|
| Primer | Oligonucleotide | Primer | Probe |
| SEQ ID NO: 3 | SEQ ID NO: 6 | SEQ ID NO: 4 | SEQ ID NO: 5 |
| SEQ ID NO: 7 | SEQ ID NO: 22 | SEQ ID NO: 8 | SEQ ID NO: 21 |
| SEQ ID NO: 7 | SEQ ID NO: 23 | SEQ ID NO: 8 | SEQ ID NO: 21 |
| SEQ ID NO: 9 | SEQ ID NO: 16 | SEQ ID NO: 10 | SEQ ID NO: 15 |
| SEQ ID NO: 9 | SEQ ID NO: 18 | SEQ ID NO: 10 | SEQ ID NO: 17 |
| SEQ ID NO: 9 | SEQ ID NO: 25 | SEQ ID NO: 10 | SEQ ID NO: 24 |
| SEQ ID NO: 9 | SEQ ID NO: 26 | SEQ ID NO: 10 | SEQ ID NO: 24 |
| SEQ ID NO: 11 | SEQ ID NO: 20 | SEQ ID NO: 12 | SEQ ID NO: 19 |

These oligonucleotide primers may also be useful for other nucleic acid amplification methods such as the ligase chain reaction (LCR) (EP 0 320 308; Carrino et al., J. Microbiol. Methods 23:3-20 (1995)); nucleic acid sequence-based amplification (NASBA) (Carrino et al., 1995, supra); and self-sustained sequence replication (3SR) and “Q replicase amplification” (Pfeffer et al., Vet. Res. Commun. 19:375-407 (1995)).

The oligonucleotide primers of the present invention can also contain a detectable label, for example a 5′ end label.

In addition, oligonucleotides of the present invention also may be used as hybridization probes. Hybridization using DNA probes has been frequently used for the detection of pathogens in food, clinical and environmental samples, and the methodologies are generally known to one skilled in the art. It is generally recognized that the degree of sensitivity and specificity of probe hybridization is lower than that achieved through the previously described amplification techniques. The nucleic acid probes of the present invention can also possess a detectable label, such as a reporter-quencher combination as are employed in Scorpion probe assays or in 5′-exonuclease detection assays, such as the Taqman® assay.

The 3′ terminal nucleotide of the nucleic acid probe may be rendered incapable of extension by a nucleic acid polymerase in one embodiment of the invention. Such blocking may be carried out, for example by the attachment of a replication inhibitor moiety, such as a reporter or quencher, to the terminal 3′ carbon of the nucleic acid probe by a linking moiety, or by making the 3′-terminal nucleotide a dideoxynucleotide. Alternatively, the 3′ end of the nucleic acid probe may be rendered impervious to the 3′ to 5′ extension activity of a polymerase by incorporating one or more modified internucleotide linkages onto the 3′ end of the oligonucleotide. Minimally, the 3′ terminal internucleotide linkage must be modified, however, additional internucleotide linkages may be modified. Internucleotide modifications which prevent elongation from the 3′ end of the nucleic acid probe and/or which block the 3′ to 5′ exonuclease activity of the DNA polymerase during PCR may include phosphorothioate linkages, methylphosphonate linkages, boranophosphate linkages, and other similar polymerase-resistant internucleotide linkages. An alternative method to block 3′ extension of the probe is to form an adduct at the 3′ end of the probe using mitomycin C or other like antitumor antibiotics such as described in Basu et al., Biochemistry 32:4708-18 (1993). Thus, the precise mechanism by which the 3′ end of the nucleic acid probe is protected from cleavage is not essential so long as the quencher is not cleaved from the nucleic acid probe.

A nucleic acid probe sequence can also optionally be employed with the primer sequence pairs of the present invention in an amplification based detection technique, such as in the 3′-exonuclease assay. Preferred primer/probe combinations are indicated in Table 1.

Preferably, SEQ ID NO:5 is 5′ end-labeled with a Quasar670 reporter and its corresponding quencher (SEQ ID NO:6) possesses a BHQ-2 label at or near the 3′ end (e.g., attached to nucleotide 30); SEQ ID NOs: 15, 17, and 19 are 5′ end-labeled with a Cal Fluor® Gold 540 reporter and their corresponding quenchers (SEQ ID NOs: 16, 18, and 20, respectively) possess a BHQ-1 label at or near the 3′ end (e.g., attached to nucleotide 26 of SEQ ID NO:16, nucleotide 30 of SEQ ID NO:18, and nucleotide 29 of SEQ ID NO:20); and SEQ ID NOs: 21 and 24 are 5′ end-labeled with a Cal Fluor® Red 610 reporter and their corresponding quenchers (SEQ ID NOs: 22 or 23 for SEQ ID NO:21 and SEQ ID NOs: 25 or 26 for SEQ ID NO:24) possess a BHQ-2 label at or near the 3′ end (e.g., attached to nucleotide 26 of SEQ ID NO:16, nucleotide 30 of SEQ ID NO:18, and nucleotide 29 of SEQ ID NO:20).

Some oligonucleotides of the present invention contain both primer and probe regions, and thus can be employed as a primer-probe complex in an appropriate assay, such as a Scorpion probe assay. These primer probe complexes of the instant invention contain a non-amplifiable linker that connects the 3′ terminus of the probe region to the 5′ terminus of the primer region. This non-amplifiable linker stops extension of a complementary strand from proceeding into the probe region of the primer-probe complex. Examples of such non-amplifiable linkages include 6-carbon linkers and preferably hexethylene glycol (HEG) linkers. Primer-probe complexes of the present invention can also contain a self-complementary region that allows the primer-probe complex to form a stem-loop structure when the probe is unbound from its target DNA, which may be useful, for example, in bringing the reporter and quencher into sufficiently close proximity to one another to cause the reporter signal to be quenched. Examples of such primer-probe complexes with self-complementary regions include SEQ ID NO:5 linked to all or a portion of SEQ ID NO:3 with a hexethylene glycol spacer, SEQ ID NO:15 linked to all or a portion of SEQ ID NOs: 9 or 11 with a hexethylene glycol spacer, SEQ ID NO:17 linked to all or a portion of SEQ ID NOs: 9 or 11 with a hexethylene glycol spacer, SEQ ID NO:19 linked to all or a portion of SEQ ID NO:12 with a hexethylene glycol spacer, SEQ ID NO:21 linked to all or a portion of SEQ ID NO:8 with an a hexethylene glycol spacer, and SEQ ID NO:24 linked to all or a portion of SEQ ID NO:10.

### Assay Methods

Detection of the presence of S. enteritidis and/or S. typhimurium, may be accomplished in any suitable manner. Preferred methods are primer-directed amplification methods and nucleic acid hybridization methods. These methods may be used to detect S. enteritidis and/or S. typhimurium in a sample that is either a complex matrix or a purified culture, e.g., from an animal, environmental, or food source suspected of contamination.

A preferred embodiment of the instant invention comprises (1) culturing a complex sample mixture in a non-selective growth media to resuscitate the target bacteria, (2) releasing total target bacterial DNA, and (3) subjecting the total DNA to an amplification protocol with a primer pair of the invention and optionally with a nucleic acid probe comprising a detectable label.

Primer-Directed Amplification Assay Methods

A variety of primer-directed nucleic acid amplification methods are known in the art which can be employed in the present invention, including thermal cycling methods (e.g., PCR, RT-PCR, and LCR), as well as isothermal methods and strand displacement amplification (SDA). The preferred method is PCR.

Sample Preparation:

The oligonucleotides and methods according to the instant invention may be used directly with any suitable clinical or environmental samples, without any need for sample preparation. In order to achieve higher sensitivity, and in situations where time is not a limiting factor, it is preferred that the samples be pre-treated and that pre-amplification enrichment is performed.

The minimum industry standard for the detection of food-borne bacterial pathogens is a method that will reliably detect the presence of one pathogen cell in 25 g of food matrix as described in Andrews et al., 1984, “Food Sample and Preparation of Sample Homogenate”, Chapter 1 in Bacteriological Analytical Manual, 8th Edition, Revision A, U.S. Food and Drug Administration. In order to satisfy this stringent criterion, enrichment methods and media have been developed to enhance the growth of the target pathogen cell in order to facilitate its detection by biochemical, immunological or nucleic acid hybridization means. Typical enrichment procedures employ media that will enhance the growth and health of the target bacteria and also inhibit the growth of any background or non-target microorganisms present.

Selective media have been developed for a variety of bacterial pathogens and one of skill in the art will know to select a medium appropriate for the particular organism to be enriched, e.g. S. enteritidis and/or S. typhimurium. A general discussion and recipes of non-selective media are described in the FDA Bacteriological Analytical Manual. (1998) published and distributed by the Association of Analytical Chemists, Suite 400, 2200 Wilson Blvd, Arlington, Va. 22201-3301.

After selective growth, a sample of the complex mixtures is removed for further analysis. This sampling procedure may be accomplished by a variety of means well known to those skilled in the art. In a preferred embodiment, 5 μl of the enrichment culture is removed and added to 200 μl of lysis solution containing protease. The lysis solution is heated at 37° C. for 20 min followed by protease inactivation at 95° C. for 10 min, and cooled to 4° C. as described in the BAX® System User's Guide, DuPont Nutrition and Health, Wilmington, Del.

PCR Assay Methods:

A preferred method for detecting the presence of S. enteritidis and/or S. typhimurium in a sample comprises (a) performing PCR amplification using primer pairs listed in Table 1 to produce a PCR amplification result; and (b) detecting the amplification, whereby a positive detection of the amplification indicates the presence of S. enteritidis and/or S. typhimurium in the sample.

In another preferred embodiment, prior to performing PCR amplification, a step of preparing the sample may be carried out. The preparing step may comprise at least one of the following processes: (1) bacterial enrichment, (2) separation of bacterial cells from the sample, (3) cell lysis, and (4) total DNA extraction.

Amplification Conditions:

A skilled person will understand that any generally acceptable PCR conditions may be used for successfully detecting S. enteritidis and/or S. typhimurium using the oligonucleotides of the instant invention, and depending on the sample to be tested and other laboratory conditions, routine optimization for the PCR conditions may be necessary to achieve optimal sensitivity and specificity. Optimally, they achieve PCR amplification results from all of the intended specific targets while giving no PCR results for other, non-target species.

Detection/Examination/Analysis:

Primer-directed amplification products can be analyzed using various methods. Homogenous detection refers to a preferred method for the detection of amplification products where no separation (such as by gel electrophoresis) of amplification products from template or primers is necessary. Homogeneous detection is typically accomplished by measuring the level of fluorescence of the reaction mixture during or immediately following amplification. In addition, heterogeneous detection methods, which involve separation of amplification products during or prior to detection, can be employed in the present invention.

Homogenous detection may be employed to carry out “real-time” primer-directed nucleic acid amplification and detection, using primer pairs of the instant invention (e.g., “real-time” PCR and “real-time” RT-PCR). Preferred “real-time” methods are set forth in U.S. Pat. Nos. 6,171,785, 5,994,056, 6,326,145, 5,804,375, 5,538,848, 5,487,972, and 5,210,015, each of which is hereby incorporated by reference in its entirety.

A particularly preferred “real-time” detection method is the Scorpion probe assay as set forth in U.S. Pat. No. 6,326,145, which is hereby incorporated by reference in its entirety. In the Scorpion probe assay, PCR amplification is performed using a Scorpion probe (either unimolecular or bimolecular) as a primer-probe complex, the Scorpion probe possessing an appropriate reporter-quencher pair to allow the detectable signal of the reporter to be quenched prior to elongation of the primer. Post-elongation, the quenching effect is eliminated and the amount of signal present is quantitated. As the amount of amplification product increases, an equivalent increase in detectable signal will be observed, thus allowing the amount of amplification product present to be determined as a function of the amount of detectable signal measured. When more than one Scorpion probe is employed in a Scorpion probe assay each probe can have a different detectable label (e.g., reporter-quencher pair) attached, thus allowing each probe to be detected independently of the other probes.

Another preferred “real-time” detection method is the 5′-exonuclease detection method, as set forth in U.S. Pat. Nos. 5,804,375, 5,538,848, 5,487,972, and 5,210,015, each of which is hereby incorporated by reference in its entirety. In the 5′-exonuclease detection assay a modified probe is employed during PCR which binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′ to 3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are preferably attached to the probe within a few nucleotides of one another, usually within 30 nucleotides of one another, more preferably with a separation of from about 6 to 16 nucleotides. Typically, this separation is achieved by attaching one member of a reporter-quencher pair to the 5′ end of the probe and the other member to a nucleotide about 6 to 16 nucleotides away.

Again, when more than one Taqman® probe is employed in a 5′-exonuclease detection assay, each probe can have a different detectable label (e.g., reporter-quencher pair) attached, thus allowing each probe to be detected independently of the other probes.

Another preferred method of homogenous detection involves the use of DNA melting curve analysis, particularly with the BAX® System hardware and reagent tablets from DuPont Nutrition and Health. The details of the system are given in U.S. Pat. No. 6,312,930 and PCT Publication Nos. WO 97/11197 and WO 00/66777, each of which is hereby incorporated by reference in its entirety.

Melting curve analysis detects and quantifies double stranded nucleic acid molecule (“dsDNA” or “target”) by monitoring the fluorescence of the target amplification product (“target amplicon”) during each amplification cycle at selected time points.

As is well known to the skilled artisan, the two strands of a dsDNA separate or melt, when the temperature is higher than its melting temperature. Melting of a dsDNA molecule is a process, and under a given solution condition, melting starts at a temperature (designated Tms hereinafter), and completes at another temperature (designated Tme hereinafter). The familiar term, Tm, designates the temperature at which melting is 50% complete.

A typical PCR cycle involves a denaturing phase where the target dsDNA is melted, a primer annealing phase where the temperature optimal for the primers to bind to the now-single-stranded target, and a chain elongation phase (at a temperature Te) where the temperature is optimal for DNA polymerase to function.

According to the present invention, Tms should be higher than Te, and Tme should be lower (often substantially lower) than the temperature at which the DNA polymerase is heat-inactivated. Melting characteristics are affected by the intrinsic properties of a given dsDNA molecule, such as deoxynucleotide composition and the length of the dsDNA.

Intercalating dyes will bind to double stranded DNA. The dye/dsDNA complex will fluoresce when exposed to the appropriate excitation wavelength of light, which is dye dependent, and the intensity of the fluorescence may be proportionate to concentration of the dsDNA. Methods taking advantage of the use of DNA intercalating dyes to detect and quantify dsDNA are known in the art. Many dyes are known and used in the art for these purposes. The instant methods also take advantage of such relationship.

Examples of such intercalating dyes include, but are not limited to, SYBR Green-I®, ethidium bromide, propidium iodide, TOTO®-1 {Quinolinium, 1-1′-[1,3-propanediylbis [(dimethyliminio)-3,1-propanediyl]]bis[4-[(3-methyl-2(3H)-benzothiazolylidene) methyl]]-, tetraiodide}, and YoPro® {Quinolinium, 4-[(3-methyl-2(3H)-benzoxazolylidene)methyl]-1-[3-(trimethylammonio)-propyl]-, diiodide}. Most preferred for the instant invention is a non-asymmetrical cyanide dye such as SYBR Green-I®, manufactured by Molecular Probes, Inc. (Eugene, Oreg.).

Melting curve analysis is achieved by monitoring the change in fluorescence while the temperature is increased. When the temperature reaches the T_{MS} specific for the target amplicon, the dsDNA begins to denature. When the dsDNA denatures, the intercalating dye dissociates from the DNA and fluorescence decreases. Mathematical analysis of the negative of the change of the log of fluorescence divided by the change in temperature plotted against the temperature results in the graphical peak known as a melting curve.

It should be understood that the present invention could be operated using a combination of these techniques, such as by having a Scorpion probe directed to one target region and a Taqman® probe directed to a second target region. It should also be understood that the invention is not limited to the above described techniques. Rather, one skilled in the art would recognize that other techniques for detecting amplification as known in the art may also be used. For example, techniques such as PCR-based quantitative sequence detection (QSD) may be performed using nucleic acid probes which, when present in the single-stranded state in solution, are configured such that the reporter and quencher are sufficiently close to substantially quench the reporter's emission. However, upon hybridization of the intact reporter-quencher nucleic acid probe with the amplified target nucleic acid sequence, the reporter and quenchers become sufficiently distant from each other. As a result, the quenching is substantially abated causing an increase in the fluorescence emission detected.

In addition to homogenous detection methods, a variety of other heterogeneous detection methods are known in the art which can be employed in the present invention, including standard non-denaturing gel electrophoresis (e.g., acrylamide or agarose), denaturing gradient gel electrophoresis, and temperature gradient gel electrophoresis. Standard non-denaturing gel electrophoresis is a simple and quick method of PCR detection, but may not be suitable for all applications.

Denaturing Gradient Gel Electrophoresis (DGGE) is a separation method that detects differences in the denaturing behavior of small DNA fragments (200-700 bp). The principle of the separation is based on both fragment length and nucleotide sequence. In fragments that are the same length, a difference as little as one base pair can be detected. This is in contrast to non-denaturing gel electrophoresis, where DNA fragments are separated only by size. This limitation of non-denaturing gel electrophoresis results because the difference in charge density between DNA molecules is near neutral and plays little role in their separation. As the size of the DNA fragment increases, its velocity through the gel decreases.

DGGE is primarily used to separate DNA fragments of the same size based on their denaturing profiles and sequence. Using DGGE, two strands of a DNA molecule separate, or melt, when heat or a chemical denaturant is applied. The denaturation of a DNA duplex is influenced by two factors: 1) the hydrogen bonds formed between complimentary base pairs (since GC rich regions melt at higher denaturing conditions than regions that are AT rich); and 2) the attraction between neighboring bases of the same strand, or “stacking”. Consequently, a DNA molecule may have several melting domains with each of their individual characteristic denaturing conditions determined by their nucleotide sequence. DGGE exploits the fact that otherwise identical DNA molecules having the same length and DNA sequence, with the exception of only one nucleotide within a specific denaturing domain, will denature at different temperatures or Tm. Thus, when the double-stranded (ds) DNA fragment is electrophoresed through a gradient of increasing chemical denaturant it begins to denature and undergoes both a conformational and mobility change. The dsDNA fragment will travel faster than a denatured single-stranded (ss) DNA fragment, since the branched structure of the single-stranded moiety of the molecule becomes entangled in the gel matrix. As the denaturing environment increases, the dsDNA fragment will completely dissociate and mobility of the molecule through the gel is retarded at the denaturant concentration at which the particular low denaturing domains of the DNA strand dissociate. In practice, the electrophoresis is conducted at a constant temperature (around 60° C.) and chemical denaturants are used at concentrations that will result in 100% of the DNA molecules being denatured (i.e., 40% formamide and 7 M urea). This variable denaturing gradient is created using a gradient maker, such that the composition of each DGGE gel gradually changes from 0% denaturant up to 100% denaturant. Of course, gradients containing a reduced range of denaturant (e.g., 35% to 60%) may also be poured for increased separation of DNA.

The principle used in DGGE can also be applied to a second method that uses a temperature gradient instead of a chemical denaturant gradient. This method is known as Temperature Gradient Gel Electrophoresis (TGGE). This method makes use of a temperature gradient to induce the conformational change of dsDNA to ssDNA to separate fragments of equal size with different sequences. As in DGGE, DNA fragments with different nucleotide sequences will become immobile at different positions in the gel. Variations in primer design can be used to advantage in increasing the usefulness of DGGE for characterization and identification of the PCR products. These methods and principles of using primer design variations are described in PCR Technology Principles and Applications, Henry A. Erlich Ed., M. Stockton Press, NY, pages 71 to 88 (1988).

Instrumentation:

When homogenous detection is employed, the level of fluorescence is preferably measured using a laser fluorometer such as, for example, BAX® Q7 machine (DuPont Nutrition and Health, Wilmington, Del.). However, similar detection systems for measuring the level of fluorescence in a sample are included in the invention.

Reagents and Kits:

Any suitable nucleic acid replication composition (“replication composition”) in any format can be used. A typical replication composition for PCR amplification may comprise, for example, dATP, dCTP, dGTP, dTTP, target specific primers and a suitable polymerase.

If the replication composition is in liquid form, suitable buffers known in the art may be used (Sambrook, J. et al., supra).

Alternatively, if the replication composition is contained in a tablet form, then typical tabletization reagents may be included such as stabilizers and binding agents. Preferred tabletization technology is set forth in U.S. Pat. Nos. 4,762,857 and 4,678,812, each of which is hereby incorporated by reference in its entirety.

A preferred replication composition of the instant invention comprises (a) the primer pair from Table 1 and (b) thermostable DNA polymerase.

A more preferred replication composition of the present invention comprises (a) the primer pairs and any corresponding probe or blocking oligonucleotide selected from Table 1, wherein each nucleic acid probe or primer-probe complex employed comprises a detectable label; and (b) thermostable DNA polymerase. Preferably the detectable label comprises a reporter capable of emitting a detectable signal and a quencher capable of substantially quenching the reporter and preventing the emission of the detectable signal when the reporter and quencher are in sufficiently close proximity to one another.

A preferred kit of the instant invention comprises any one of the above replication compositions. A preferred tablet of the instant invention comprises any one of the above replication compositions. More preferably, a kit of the instant invention comprises the foregoing preferred tablet.

In some instances, an internal positive control can be included in the reaction. The internal positive control can include control template nucleic acids (e.g. DNA or RNA), control primers, and control nucleic acid probe. The advantages of an internal positive control contained within a PCR reaction have been previously described (U.S. Pat. No. 6,312,930 and PCT Application No. WO 97/11197, each of which is hereby incorporated by reference in its entirety), and include: (i) the control may be amplified using a single primer; (ii) the amount of the control amplification product is independent of any target DNA or RNA contained in the sample; (iii) the control DNA can be tableted with other amplification reagents for ease of use and high degree of reproducibility in both manual and automated test procedures; (iv) the control can be used with homogeneous detection, i.e., without separation of product DNA from reactants; and (v) the internal control has a melting profile that is distinct from other potential amplification products in the reaction and/or a detectable label on the control nucleic acid that is distinct from the detectable label on the nucleic acid probe directed to the target.

Control DNA will be of appropriate size and base composition to permit amplification in a primer-directed amplification reaction. The control template DNA sequence may be obtained from the S. enteritidis or S. typhimurium genome, or from another source, but must be reproducibly amplified under the same conditions that permit the amplification of the target amplification product.

Preferred control sequences include, for example, those found in SV40 (SEQ ID NO:27). The preferred concentration range of SV40, when used, is 10¹ to 10⁷ copies per PCR reaction.

The control reaction is useful to validate the amplification reaction. Amplification of the control DNA occurs within the same reaction tube as the sample that is being tested, and therefore indicates a successful amplification reaction when samples are target negative, i.e. no target amplification product is produced. In order to achieve significant validation of the amplification reaction, a suitable number of copies of the control DNA template must be included in each amplification reaction.

In some instances it may be useful to include an additional negative control replication composition. The negative control replication composition will contain the same reagents as the replication composition but without the polymerase. The primary function of such a control is to monitor spurious background fluorescence in a homogeneous format when the method employs a fluorescent means of detection.

Replication compositions may be modified depending on whether they are designed to be used to amplify target DNA or the control DNA. Replication compositions that will amplify the target DNA (test replication compositions) may include (i) a polymerase (generally thermostable), (ii) a primer pair capable of hybridizing to the target DNA and (iii) necessary buffers for the amplification reaction to proceed. Replication compositions that will amplify the control DNA (positive control, or positive replication composition) may include (i) a polymerase (generally thermostable) (ii) the control DNA; (iii) at least one primer capable of hybridizing to the control DNA; and (iv) necessary buffers for the amplification reaction to proceed. In addition, the replication composition for either target DNA or control DNA amplification can contain a nucleic acid probe, preferably possessing a detectable label.

Nucleic Acid Hybridization Methods

In addition to primer-directed amplification assay methods, nucleic acid hybridization assay methods can be employed in the present invention for detection of S. enteritidis and/or S. typhimurium. The basic components of a nucleic acid hybridization test include probe(s), a sample suspected of containing S. enteritidis and/or S. typhimurium, and a specific hybridization method. Typically the probe(s) length can vary from as few as five bases to the full length of the S. enteritidis or S. typhimurium diagnostic sequence and will depend upon the specific test to be done. Only part of the probe molecule need be complementary to the nucleic acid sequence to be detected. In addition, the complementarity between the probe(s) and the target sequence(s) need not be perfect. Hybridization does occur between imperfectly complementary molecules with the result that a certain fraction of the bases in the hybridized region(s) are not paired with the proper complementary base.

Probes particularly useful in nucleic acid hybridization methods are any of SEQ ID NOs: 5, 15, 17, 19, 21, or 24, or sequences derived therefrom.

The sample may or may not contain S. enteritidis or S. typhimurium. The sample may take a variety of forms, however will generally be extracted from an animal, environmental or food source suspected of contamination. The DNA may be detected directly but most preferably, the sample nucleic acid must be made available to contact the probe before any hybridization of probe(s) and target molecule(s) can occur. Thus the organism's DNA is preferably free from the cell and placed under the proper conditions before hybridization can occur. Methods of in-solution hybridization necessitate the purification of the DNA in order to be able to obtain hybridization of the sample DNA with the probe(s). This has meant that utilization of the in-solution method for detection of target sequences in a sample requires that the nucleic acids of the sample must first be purified to eliminate protein, lipids, and other cell components, and then contacted with the probe(s) under hybridization conditions. Methods for the purification of the sample nucleic acid are common and well known in the art (Sambrook et al., supra).

In one preferred embodiment, hybridization assays may be conducted directly on cell lysates, without the need to extract the nucleic acids. This eliminates several steps from the sample-handling process and speeds up the assay. To perform such assays on crude cell lysates, a chaotropic agent is typically added to the cell lysates prepared as described above. The chaotropic agent stabilizes nucleic acids by inhibiting nuclease activity. Furthermore, the chaotropic agent allows sensitive and stringent hybridization of short oligonucleotide probes to DNA at room temperature (Van Ness & Chen, Nucleic Acids Res. 19:5143-51 (1991)). Suitable chaotropic agents include guanidinium chloride, guanidinium thiocyanate, sodium thiocyanate, lithium tetrachloroacetate, sodium perchlorate, rubidium tetrachloroacetate, potassium iodide, and cesium trifluoroacetate, among others. Typically, the chaotropic agent will be present at a final concentration of about 3 M. If desired, one can add formamide to the hybridization mixture, typically 30-50% (v/v).

Alternatively, one can purify the sample nucleic acids prior to probe hybridization. A variety of methods are known to one of skill in the art (e.g., phenol-chloroform extraction, IsoQuick extraction (MicroProbe Corp., Bothell, Wash.), and others). Pre-hybridization purification is particularly useful for standard filter hybridization assays. Furthermore, purification facilitates measures to increase the assay sensitivity by incorporating in vitro RNA amplification methods such as self-sustained sequence replication (see for example Fahy et al., In PCR Methods and Applications, Cold Spring Harbor Laboratory: Cold Spring Harbor, N.Y. (1991), pp. 25-33) or reverse transcriptase PCR (Kawasaki, In PCR Protocols: A Guide to Methods and Applications, M. A. Innis et al., Eds., (1990), pp. 21-27).

Once the DNA is released, it can be detected by any of a variety of methods. However, the most useful embodiments have at least some characteristics of speed, convenience, sensitivity, and specificity.

Hybridization methods are well known in the art. Typically the probe and sample must be mixed under conditions which will permit nucleic acid hybridization. This involves contacting the probe and sample in the presence of an inorganic or organic salt under the proper concentration and temperature conditions. The probe and sample nucleic acids must be in contact for a long enough time that any possible hybridization between the probe and sample nucleic acid may occur. The concentration of probe or target in the mixture will determine the time necessary for hybridization to occur. The higher the probe or target concentration, the shorter the hybridization incubation time needed.

Various hybridization solutions can be employed. Typically, these comprise from about 20 to 60% volume, preferably 30%, of a polar organic solvent. A common hybridization solution employs about 30-50% v/v formamide, about 0.15 to 1M sodium chloride, about 0.05 to 0.1M buffers, such as sodium citrate, Tris-HCl, PIPES or HEPES (pH range about 6-9), about 0.05 to 0.2% detergent, such as sodium dodecylsulfate, or between 0.5-20 mM EDTA, FICOLL (Pharmacia Inc.) (about 300-500 kilodaltons), polyvinylpyrrolidone (about 250-500 kdal), and serum albumin. Also included in the typical hybridization solution will be unlabeled carrier nucleic acids from about 0.1 to 5 mg/mL, fragmented nucleic DNA (e.g., calf thymus or salmon sperm DNA, or yeast RNA), and optionally from about 0.5 to 2% wt/vol glycine. Other additives may also be included, such as volume exclusion agents which include a variety of polar water-soluble or swellable agents (e.g., polyethylene glycol), anionic polymers (e.g., polyacrylate or polymethylacrylate), and anionic saccharidic polymers (e.g., dextran sulfate).

Nucleic acid hybridization is adaptable to a variety of assay formats. One of the most suitable is the sandwich assay format. The sandwich assay is particularly adaptable to hybridization under non-denaturing conditions. A primary component of a sandwich-type assay is a solid support. The solid support has adsorbed to it or covalently coupled to it immobilized nucleic acid probe that is unlabeled and complementary to one portion of the DNA sequence.

The sandwich assay may be encompassed in an assay kit. This kit would include a first component for the collection of samples suspected of contamination and buffers for the disbursement and lysis of the sample. A second component would include media in either dry or liquid form for the hybridization of target and probe polynucleotides, as well as for the removal of undesirable and nonduplexed forms by washing. A third component includes a solid support (dipstick) upon which is fixed (or to which is conjugated) unlabeled nucleic acid probe(s) that is (are) complementary to one or more of the sequences disclosed herein. A fourth component would contain labeled probe that is complementary to a second and different region of the same DNA strand to which the immobilized, unlabeled nucleic acid probe of the third component is hybridized.

In a preferred embodiment, polynucleotide sequences disclosed herein or derivations thereof may be used as 3′ blocked detection probes in either a homogeneous or heterogeneous assay format. For example, a probe generated from these sequences may be 3′ blocked or non-participatory and will not be extended by, or participate in, a nucleic acid amplification reaction. Additionally, the probe incorporates a label that can serve as a reactive ligand that acts as a point of attachment for the immobilization of the probe/analyte hybrid or as a reporter to produce detectable signal. Accordingly, genomic or cDNA isolated from a sample suspected of S. enteritidis and/or S. typhimurium contamination is amplified by standard primer-directed amplification protocols in the presence of an excess of the 3′ blocked detection probe(s) to produce amplification products. Because the probe(s) is 3′ blocked, it does not participate or interfere with the amplification of the target. After the final amplification cycle, the detection probe(s) anneals to the relevant portion of the amplified DNA and the annealed complex is then captured on a support through the reactive ligand.

In some instances it is desirable to incorporate a ligand labeled dNTP, with the label probe in the replication composition to facilitate immobilization of the PCR reaction product on a support and then detection of the immobilized product by means of the labeled probe reagent. For example a biotin, digoxigenin, or digoxin labeled dNTP could be added to PCR reaction composition. The biotin, digoxigenin, or digoxin incorporated in the PCR product could then be immobilized respectively on to a strepavidin, anti-dixogin or antidigoxigenin antibody support. The immobilized PCR product could then be detected by the presence of the probe label.

### EXAMPLES

The present invention is further defined in the following Examples. It should be understood that these Examples, while indicating preferred embodiments of the invention, are given by way of illustration only.

General Methods and Materials Used in the Examples

Materials and methods suitable for the maintenance and growth of bacterial cultures are well known in the art. Techniques suitable for use in the following Examples may be found in Manual of Methods for Genus Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994) or Thomas D. Brock in Biotechnology: A Textbook of Industrial Microbiology, Second Edition (1989) Sinauer Associates, Inc., Sunderland, Mass. or Bacteriological Analytical Manual. 6th Edition, Association of Official Analytical Chemists, Arlington, Va. (1984).

Primers and probes (SEQ ID NOs: 3-26) were prepared by Biosearch Technologies, Inc., 2199 S. McDowell Blvd., Petaluma, Calif. 94954 USA.

All PCR reactions were carried out using a standard BAX® System (DuPont Nutrition and Health, Wilmington, Del.).

The meaning of abbreviations is as follows: “h” means hour(s), “min” means minute(s), “sec” means second(s), “d” means day(s), “ml” means milliliter(s), “μl” means microliter(s), “cfu” means colony forming unit(s), “M” means molar, “μM” means micromolar, “nM” means nanomolar, “SE” means Salmonella enteritidis, “ST” means Salmonella typhimurium, “Ct” means cycle threshold, “IPC” means internal positive control.

### Example 1

### Development of S. enteritidis Primers and Scorpion Probes

Analysis of PCR products in gel indicated amplification of S. enteritidis targets at two different primer concentrations, 100 nM and 400 nM, was observed. Then three candidate biomolecular Scorpion probes with their complementary full-length (N-0) and shorter-length (N-3) quencher were evaluated for sensitivity and reaction dynamics using primers SEQ ID NOs: 3 and 4. The results showed that, using a dilution series of S. enteritidis lysates plus water as a negative control, SEQ ID NO:5 with its full length quencher, SEQ ID NO:6, yielded the strongest signal response (in terms of both amplitude and potential Ct value) compared to the other probe/quencher combinations. In addition, inter-replacement of unlabeled primer and Scorpion probe showed no significant on target amplification.

Probe and quencher concentrations were also titrated from 10 nM to 60 nM for the probe, and 20 nM to 120 nM for the quencher. Based on the probe titration results, 20 nM probe with 40 nM quencher showed the best PCR and Scorpion performance in terms of cleavage kinetics and lower Ct values. Under the same condition, the single-plexed S. enteritidis assay was able to detect 10⁴ cfu/mL of S. enteritidis in triplicates.

### Example 2

### Development of S. typhimurium Primers and Scorpion Probes

Using a serial dilution of S. typhimurium templates and negative controls, performance of S. typhimurium Scorpion probes and the complementary quenchers were evaluated using primers SEQ ID NOs: 7 and 8. In the initial evaluation, out of five potential probe:quencher pairing opportunities, the pairs SEQ ID NO:17/SEQ ID NO:18 and SEQ ID NO:17/SEQ ID NO:16 showed better performance in terms of low Ct values and relatively good fluorescence dynamic range. When concentrations of the selected Scorpion probes and quenchers were varied to determine an optimal reaction condition, it was determined based on Ct values and fluorescence intensities, that when SEQ ID NO:15/SEQ ID NO:18 were spiked at 20/40 and 25/50 nM, this primer/probe pair yielded the best results.

Molar ratios of SEQID NO:15/SEQ ID NO:18 were tested at 1:0.5, 1:1, 1:1.5, 1:2, 1:2.5 and 1:3 when the probe concentration was maintained at 25 nM. The results showed that best quenching efficiency was observed when the probe:quencher molar ratios were 1:2, 1:2.5 and 1:3. Using 25 nM probe and 50 nM quencher, the assay was able to show a sensitivity of 10⁴ cfu/mL of S. typhimurium.

### Example 3

### Multiplexing Assay

When 150 nM of S. typhimurium primers, SEQ ID NO:7 and SEQ ID NO:8, and 400 nM of S. enteritidis primers, SEQ ID NO:3 and SEQ ID NO:4, were combined, no negative effect was observed in terms of their performance. In addition, no cross dimer was found.

Then S. enteritidis and S. typhimurium scorpion assays were combined and tested separately by S. enteritidis templates and S. typhimurium templates, or both. The results showed that S. typhimurium primers, SEQ ID NO:7 and SEQ ID NO:8, S. typhimurium probe SEQ ID NO:21 and S. typhimurium quencher SEQ ID NO:22 were compatible with S. enteritidis primers, SEQ ID NO:3 and SEQ ID NO:4, S. enteritidis probe SEQ ID NO:5 and S. enteritidis quencher SEQ ID NO:6. In addition, increase of S. enteritidis primers from 400 nM to 600 nM under the multiplexed conditions also improved detection of low titer S. enteritidis templates in terms of recovery rate and amplification kinetics.

Under the multiplexing condition, the assay was able to detect S. enteritidis and S. typhimurium targets down to ˜7×10³ cfu/mL. When both targets were present in the same reaction, the assay showed a sensitivity of ˜3.5×10³ cfu/mL for both S. enteritidis and S. typhimurium. With all titer dilutions, consistent performance of IPC was observed. Consistent and optimal performance was achieved with 150 nM of S. typhimurium primers (SEQ ID NOs: 7 and 8) and 600 nM of S. enteritidis primers (SEQ ID NOs: 3 and 4).

### Example 4

### Inclusivity Assays

A total of 31 strains of S. enteritidis from the internal DuPont Nutrition & Health culture collection (designated as “DD”) and US FDA culture collection (designated as “SAFE” or “SARA”) were grown in Brain-Heart Infusion Broth (BHI) overnight at 37° C. DNA was extracted using the standard BAX lysis protocol, and tested without dilution by the single-plexed S. enteritidis scorpion. All showed positive signals (Table 2). Among them, 24 strains (#5 to #28 listed in Table 2) were further diluted to approximately 10⁴ cfu/mL and tested by the multiplexing assay. All strains were tested positive.

Similarly, to validate target of detection for S. typhimurium, 57 strains of S. typhimurium including the monophasic variant, Salmonella 4,5,12:i:—, from the DD and SAFE collections were also tested without dilution by the single-plexed S. typhimurium scorpion. All showed positive signals (Table 3). Then, all these strains were diluted to approximately 10⁴ cfu/mL and tested by the multiplexing assay. All strains were tested positive.

TABLE 2

| # | ID | Strain | SE Test | ST Test |
|---|---|---|---|---|
| 1 | DD706 | Salmonella enteritidis | POS | NEG |
| 2 | DD1243 | Salmonella enteritidis | POS | NEG |
| 3 | DD4022 | Salmonella enteritidis | POS | NEG |
| 4 | DD6696 | Salmonella enteritidis | POS | NEG |
| 5 | SAFE 2 | Salmonella enteritidis | POS | NEG |
| 6 | SAFE 79 | Salmonella enteritidis | POS | NEG |
| 7 | SAFE 80 | Salmonella enteritidis | POS | NEG |
| 8 | SAFE 81 | Salmonella enteritidis | POS | NEG |
| 9 | DD13170 | Salmonella enteritidis | POS | NEG |
| 10 | DD13171 | Salmonella enteritidis | POS | NEG |
| 11 | DD13321 | Salmonella enteritidis | POS | NEG |
| 12 | DD13322 | Salmonella enteritidis | POS | NEG |
| 13 | DD13323 | Salmonella enteritidis | POS | NEG |
| 14 | DD13324 | Salmonella enteritidis | POS | NEG |
| 15 | DD13325 | Salmonella enteritidis | POS | NEG |
| 16 | DD13326 | Salmonella enteritidis | POS | NEG |
| 17 | DD13327 | Salmonella enteritidis | POS | NEG |
| 18 | DD13328 | Salmonella enteritidis | POS | NEG |
| 19 | DD13329 | Salmonella enteritidis | POS | NEG |
| 20 | DD13330 | Salmonella enteritidis | POS | NEG |
| 21 | DD13331 | Salmonella enteritidis | POS | NEG |
| 22 | DD13332 | Salmonella enteritidis | POS | NEG |
| 23 | DD13333 | Salmonella enteritidis | POS | NEG |
| 24 | DD13334 | Salmonella enteritidis | POS | NEG |
| 25 | DD13335 | Salmonella enteritidis | POS | NEG |
| 26 | DD13336 | Salmonella enteritidis | POS | NEG |
| 27 | DD13337 | Salmonella enteritidis | POS | NEG |
| 28 | DD13338 | Salmonella enteritidis | POS | NEG |
| 29 | DD13339 | Salmonella enteritidis | POS | NEG |
| 30 | DD13340 | Salmonella enteritidis | POS | NEG |
| 31 | DD13342 | Salmonella enteritidis | POS | NEG |

TABLE 3

| # | ID | Strain | SE Test | ST Test |
|---|---|---|---|---|
| 1 | SAFE 4 | Salmonella typhimurium | NEG | POS |
| 2 | SAFE 56 | Salmonella typhimurium | NEG | POS |
| 3 | SAFE 57 | Salmonella typhimurium | NEG | POS |
| 4 | SAFE 58 | Salmonella typhimurium | NEG | POS |
| 5 | SAFE 59 | Salmonella typhimurium | NEG | POS |
| 6 | SAFE 60 | Salmonella typhimurium | NEG | POS |
| 7 | SAFE 61 | Salmonella typhimurium | NEG | POS |
| 8 | SAFE 62 | Salmonella typhimurium | NEG | POS |
| 9 | SAFE 63 | Salmonella typhimurium | NEG | POS |
| 10 | SAFE 64 | Salmonella typhimurium | NEG | POS |
| 11 | SAFE 65 | Salmonella typhimurium | NEG | POS |
| 12 | SAFE 66 | Salmonella typhimurium | NEG | POS |
| 13 | SAFE 67 | Salmonella typhimurium | NEG | POS |
| 14 | SAFE 68 | Salmonella typhimurium | NEG | POS |
| 15 | SAFE 69 | Salmonella typhimurium | NEG | POS |
| 16 | SAFE 70 | Salmonella typhimurium | NEG | POS |
| 17 | SAFE 71 | Salmonella typhimurium | NEG | POS |
| 18 | SAFE 72 | Salmonella typhimurium | NEG | POS |
| 19 | SARA 1 | Salmonella typhimurium | NEG | POS |
| 20 | SARA 2 | Salmonella typhimurium | NEG | POS |
| 21 | SARA 3 | Salmonella typhimurium | NEG | POS |
| 22 | SARA 4 | Salmonella typhimurium | NEG | POS |
| 23 | SARA 5 | Salmonella typhimurium | NEG | POS |
| 24 | SARA 6 | Salmonella typhimurium | NEG | POS |
| 25 | SARA 7 | Salmonella typhimurium | NEG | POS |
| 26 | SARA 8 | Salmonella typhimurium | NEG | POS |
| 27 | SARA 9 | Salmonella typhimurium | NEG | POS |
| 28 | SARA 10 | Salmonella typhimurium | NEG | POS |
| 29 | SARA 11 | Salmonella typhimurium | NEG | POS |
| 30 | SARA 12 | Salmonella typhimurium | NEG | POS |
| 31 | SARA 13 | Salmonella typhimurium | NEG | POS |
| 32 | SARA 14 | Salmonella typhimurium | NEG | POS |
| 33 | SARA 15 | Salmonella typhimurium | NEG | POS |
| 34 | SARA 16 | Salmonella typhimurium | NEG | POS |
| 35 | SARA 17 | Salmonella typhimurium | NEG | POS |
| 36 | SARA 18 | Salmonella typhimurium | NEG | POS |
| 37 | SARA 19 | Salmonella typhimurium | NEG | POS |
| 38 | SARA 20 | Salmonella typhimurium | NEG | POS |
| 39 | SARA 21 | Salmonella typhimurium | NEG | POS |
| 40 | DD586 | Salmonella typhimurium | NEG | POS |
| 41 | DD1084 | Salmonella typhimurium | NEG | POS |
| 42 | DD1467 | Salmonella typhimurium | NEG | POS |
| 43 | DD1775 | Salmonella typhimurium | NEG | POS |
| 44 | DD5868 | Salmonella typhimurium | NEG | POS |
| 45 | DD6209 | Salmonella typhimurium | NEG | POS |
| 46 | DD13005 | Salmonella typhimurium | NEG | POS |
| 47 | DD13011 | Salmonella typhimurium | NEG | POS |
| 48 | DD13265 | Salmonella typhimurium | NEG | POS |
| 49 | DD13266 | Salmonella typhimurium | NEG | POS |
| 50 | DD13404 | Salmonella typhimurium | NEG | POS |
| 51 | DD13557 | Salmonella typhimurium | NEG | POS |
| 52 | SAFE 73 | Salmonella 4,5,12:i:- | NEG | POS |
| 53 | SAFE 74 | Salmonella 4,5,12:i:- | NEG | POS |
| 54 | SAFE 75 | Salmonella 4,5,12:i:- | NEG | POS |
| 55 | SAFE 76 | Salmonella 4,5,12:i:- | NEG | POS |
| 56 | SAFE 77 | Salmonella 4,5,12:i:- | NEG | POS |
| 57 | SAFE 78 | Salmonella 4,5,12:i:- | NEG | POS |

### Example 5

### Exclusivity Assays

Thirty strains of closely-related non-Salmonella strains were grown in BHI overnight at 37° C. DNA was extracted using the standard BAX lysis protocol, and tested without dilution by the multiplexing assay. All were tested negative.

More than 130 strains of non-enteritidis and non-typhimurium Salmonella were also tested by the single-plexed S. enteritidis Scorpion or single-plexed S. typhimurium (or S. typhimurium primers alone). All strains tested negative for S. enteritidis and S. typhimurium (Table 4).

TABLE 4

| # | DD# | Strain | SE/ST Multiplexing Test |
|---|---|---|---|
| 1 | 584 | Salmonella typhi | NEG |
| 2 | 707 | Salmonella Newport | NEG |
| 3 | 732 | Salmonella infantis | NEG |
| 4 | 741 | Salmonella gallinarum | NEG |
| 5 | 900 | Salmonella infantis | NEG |
| 6 | 917 | Salmonella choleraesuis | NEG |
| 7 | 918 | Salmonella paratyphi | NEG |
| 8 | 1085 | Salmonella binza | NEG |
| 9 | 1254 | Salmonella kedougou | NEG |
| 10 | 1254 | Salmonella kedougou | NEG |
| 11 | 1261 | Salmonella newport | NEG |
| 12 | 1329 | Salmonella braenderup | NEG |
| 13 | 1332 | Salmonella anatum | NEG |
| 14 | 1338 | Salmonella brandenburg | NEG |
| 15 | 1343 | Salmonella haardt | NEG |
| 16 | 1356 | Salmonella bredeney | NEG |
| 17 | 1370 | Salmonella stanley | NEG |
| 18 | 1372 | Salmonella St Paul | NEG |
| 19 | 1424 | Salmonella manchester | NEG |
| 20 | 1428 | Salmonella frintrop | NEG |
| 21 | 1428 | Salmonella frintrop | NEG |
| 22 | 1429 | Salmonella anfo | NEG |
| 23 | 1474 | Salmonella havana | NEG |
| 24 | 1476 | Salmonella napoli | NEG |
| 25 | 1480 | Salmonella indiana | NEG |
| 26 | 1490 | Salmonella panama | NEG |
| 27 | 1491 | Salmonella weltevreden | NEG |
| 28 | 1507 | Salmonella Pullorum | NEG |
| 29 | 1509 | Salmonella bovismorbificans | NEG |
| 30 | 1510 | Salmonella bareilly | NEG |
| 31 | 1521 | Salmonella abaetetuba | NEG |
| 32 | 1523 | Salmonella berkeley | NEG |
| 33 | 1523 | Salmonella berkeley | NEG |
| 34 | 1535 | Salmonella Brookfield | NEG |
| 35 | 1552 | Salmonella alabama | NEG |
| 36 | 1553 | Salmonella ball | NEG |
| 37 | 1555 | Salmonella brancaster | NEG |
| 38 | 1556 | Salmonella alachua | NEG |
| 39 | 1557 | Salmonella chicago | NEG |
| 40 | 1560 | Salmonella Westpark | NEG |
| 41 | 1585 | Salmonella arizonae | NEG |
| 42 | 1608 | Salmonella seminole | NEG |
| 43 | 1609 | Salmonella wassennaar | NEG |
| 44 | 1611 | Salmonella kralendyk | NEG |
| 45 | 1616 | Salmonella houten | NEG |
| 46 | 1616 | Salmonella houten | NEG |
| 47 | 1620 | Salmonella carmel | NEG |
| 48 | 1621 | Salmonella carrau | NEG |
| 49 | 1624 | Salmonella chandans | NEG |
| 50 | 1635 | Salmonella daytona | NEG |
| 51 | 1638 | Salmonella derby | NEG |
| 52 | 1652 | Salmonella london | NEG |
| 53 | 1653 | Salmonella yovokome | NEG |
| 54 | 1657 | Salmonella reading | NEG |
| 55 | 1658 | Salmonella schwarzengrund | NEG |
| 56 | 1659 | Salmonella shangani | NEG |
| 57 | 1660 | Salmonella sundsvall | NEG |
| 58 | 1665 | Salmonella colombo | NEG |
| 59 | 1668 | Salmonella california | NEG |
| 60 | 1675 | Salmonella salamae | NEG |
| 61 | 1680 | Salmonella dugbe | NEG |
| 62 | 1684 | Salmonella emmastad | NEG |
| 63 | 1686 | Salmonella fayed | NEG |
| 64 | 1687 | Salmonella ferlac | NEG |
| 65 | 1695 | Salmonella johannesburg | NEG |
| 66 | 1698 | Salmonella madelia | NEG |
| 67 | 1700 | Salmonella meleagridis | NEG |
| 68 | 1701 | Salmonella miami | NEG |
| 69 | 1705 | Salmonella muenster | NEG |
| 70 | 1707 | Salmonella newbrunswick | NEG |
| 71 | 1710 | Salmonella oranienburg | NEG |
| 72 | 1712 | Salmonella pretoria | NEG |
| 73 | 1773 | Salmonella bongori | NEG |
| 74 | 1896 | Salmonella hadar | NEG |
| 75 | 1897 | Salmonella hadar | NEG |
| 76 | 1899 | Salmonella hadar | NEG |
| 77 | 2179 | Salmonella infantis | NEG |
| 78 | 2180 | Salmonella champaign | NEG |
| 79 | 2186 | Salmonella drypool | NEG |
| 80 | 2189 | Salmonella give | NEG |
| 81 | 2196 | Salmonella kiambu | NEG |
| 82 | 2204 | Salmonella minnesota | NEG |
| 83 | 2205 | Salmonella mississippi | NEG |
| 84 | 2215 | Salmonella poona | NEG |
| 85 | 2238 | Salmonella urbana | NEG |
| 86 | 2239 | Salmonella cerro | NEG |
| 87 | 2263 | Salmonella lille | NEG |
| 88 | 2289 | Salmonella rubislaw | NEG |
| 89 | 2290 | Salmonella hartford | NEG |
| 90 | 2296 | Salmonella infantis | NEG |
| 91 | 2312 | Salmonella kottbus | NEG |
| 92 | 2313 | Salmonella wandsworth | NEG |
| 93 | 2341 | Salmonella mbandaka | NEG |
| 94 | 2342 | Salmonella virchow | NEG |
| 95 | 2346 | Salmonella vietnam | NEG |
| 96 | 2352 | Salmonella saphra | NEG |
| 97 | 2353 | Salmonella kristianstad | NEG |
| 98 | 2380 | Salmonella sya | NEG |
| 99 | 2639 | Salmonella thomasville | NEG |
| 100 | 2673 | Salmonella manhattan | NEG |
| 101 | 2735 | Salmonella ohio | NEG |
| 102 | 2869 | Salmonella durham | NEG |
| 103 | 2935 | Salmonella sandiego | NEG |
| 104 | 2966 | Salmonella albany | NEG |
| 105 | 2980 | Salmonella arkansas | NEG |
| 106 | 3019 | Salmonella dublin | NEG |
| 107 | 3038 | Salmonella krefeld | NEG |
| 108 | 3156 | Salmonella muenchen | NEG |
| 109 | 3184 | Salmonella sculcoates | NEG |
| 110 | 3185 | Salmonella bellevue | NEG |
| 111 | 3194 | Salmonella stanleyville | NEG |
| 112 | 3217 | Salmonella cotham | NEG |
| 113 | 3218 | Salmonella agama | NEG |
| 114 | 3432 | Salmonella amager | NEG |
| 115 | 3806 | Salmonella havana | NEG |
| 116 | 3861 | Salmonella hadar | NEG |
| 117 | 3868 | Salmonella infantis | NEG |
| 118 | 3869 | Salmonella infantis | NEG |
| 119 | 3902 | Salmonella infantis | NEG |
| 120 | 3916 | Salmonella hadar | NEG |
| 121 | 3917 | Salmonella hadar | NEG |
| 122 | 3918 | Salmonella hadar | NEG |
| 123 | 3984 | Salmonella java | NEG |
| 124 | 4035 | Salmonella waycross | NEG |
| 125 | 4044 | Salmonella hadar | NEG |
| 126 | 4055 | Salmonella virchow | NEG |
| 127 | 4057 | Salmonella infantis | NEG |
| 128 | 4102 | Salmonella StPaul | NEG |
| 129 | 4558 | Salmonella redlands | NEG |
| 130 | 5533 | Salmonella infantis | NEG |
| 131 | 6250 | Salmonella santiago | NEG |
| 132 | 6686 | Salmonella infantis | NEG |
| 133 | 6729 | Salmonella manila | NEG |
| 134 | 7050 | Salmonella virchow | NEG |
| 135 | 7061 | Salmonella kubacha | NEG |
| 136 | 7072 | Salmonella amsterdam | NEG |
| 137 | 12912 | Salmonella Kentucky | NEG |
| 138 | 12918 | Salmonella Kentucky | NEG |

### Example 6

### Food Enrichment Testing

Sensitivity and specificity of the assays were also evaluated with ground turkey enrichment. Briefly, 25 g of Salmonella-free ground turkey was enriched in 225 mL of pre-warmed Tryptic Soy Broth and incubated at 42° C. for 14 hours. 250 μL of enrichment was then mixed with 10 mL of 1×BAX lysis buffer with protease and lysed according to the standard BAX lysis protocol. S. enteritidis (strain SAFE 2) and S. typhimurium (strain SAFE 4) grown overnight in BHI at 37° C. were lysed using the standard BAX lysis protocol and then diluted in a 10-fold serial dilution using the blank turkey enrichment lysates. For detection, 25 μL of diluted lysates was mixed with 5 μL of multiplexed PCR mix. The assay showed a sensitivity of about 10⁴ cfu/mL of S. enteritidis and S. typhimurium in ground turkey enrichment lysates. No cross-reaction to the background microflora was found as all blank turkey enrichment lysates were tested negative by the assay.

1. A method for detecting the presence of Salmonella enteritidis and/or Salmonella typhimurium in a sample, said sample comprising nucleic acids, said method comprising:
(a) providing a reaction mixture comprising suitable primer pairs for amplification of at least a portion of
(i) a Salmonella enteritidis SEN0908A/SEN0909/SEN0910 region, and/or
(ii) a Salmonella typhimurium type II restriction enzyme methylase region;
(b) performing PCR amplification of said nucleic acids of said sample using the reaction mixture of step (a); and
(c) detecting the amplification of step (b), whereby a positive detection of amplification indicates the presence of Salmonella enteritidis and/or Salmonella typhimurium in the sample.

2. The method of claim 1, wherein step (a)(i) comprises suitable primer pairs for amplification of SEQ ID NO:1.

3. The method of claim 2, wherein said primer pair for amplification of the nucleic acid region of SEQ ID NO:1 comprises SEQ ID NO:3 and SEQ ID NO:4.

4. The method of claim 1, wherein step (a)(ii) comprises suitable primers pairs for amplification of SEQ ID NO:2.

5. The method of claim 4, wherein said primer pair for amplification of the nucleic acid region of SEQ ID NO:2 comprises a first nucleic acid sequence selected from the group consisting of SEQ ID NO:7, SEQ ID NO:9, SEQ ID NO:11, and SEQ ID NO:13 and a second nucleic acid sequence selected from the group consisting of SEQ ID NO:8, SEQ ID NO:10, SEQ ID NO:12, and SEQ ID NO:14.

6. The method of claim 3, wherein said reaction mixture further comprises at least one nucleic acid probe for each nucleic acid region to be detected.

7. The method of claim 6, wherein said at least one nucleic acid probe comprises one or more of SEQ ID NO:5, SEQ ID NO:15, SEQ ID NO:17, SEQ ID NO:19, SEQ ID NO:21, and SEQ ID NO:24.

8. The method of claim 7, wherein each of said at least one nucleic acid probe comprises a detectable label.

9. The method of claim 8, wherein said reaction mixture further comprises a blocking oligonucleotide capable of quenching said detectable label of said at least one nucleic acid probe comprising SEQ ID NO:6, SEQ ID NO:16, SEQ ID NO:18, SEQ ID NO:20, SEQ ID NO:22, SEQ ID NO:23, SEQ ID NO:25 or SEQ ID NO:26.

10. The method of claim 1, wherein the sample comprises a food sample or an environmental sample.

11. A primer comprising a polynucleotide sequence having at least 95% sequence identity based on the BLASTN method of alignment to the polynucleotide sequence set forth in SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:28, SEQ ID NO:29, SEQ ID NO:30, SEQ ID NO:31, SEQ ID NO:32, or SEQ ID NO:33.

12. The primer of claim 11, wherein the primer comprises the polynucleotide sequence set forth in SEQ ID NO:3, SEQ ID NO:4, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:28, SEQ ID NO:29, SEQ ID NO:30, SEQ ID NO:31, SEQ ID NO:32, or SEQ ID NO:33.

13. A probe/quencher pair comprising polynucleotide sequences having at least 95% sequence identity based on the BLASTN method of alignment to the polynucleotide sequences set forth in SEQ ID NO:5 and SEQ ID NO:6, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, or SEQ ID NO:24 and SEQ ID NO:26.

14. The probe/quencher pair of claim 13, wherein the probe/quencher pair comprises the polynucleotide nucleotide sequences set forth in SEQ ID NO:5 and SEQ ID NO:6, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, or SEQ ID NO:24 and SEQ ID NO:26.

15. A Salmonella enteritidis or Salmonella typhimurium detection sequence comprising a polynucleotide sequence having at least 95% sequence identity based on the BLASTN method of alignment to the polynucleotide sequence set forth in SEQ ID NO:1 or SEQ ID NO:2.

16. The Salmonella enteritidis or Salmonella typhimurium detection sequence of claim 15 comprising the polynucleotide sequence set forth in SEQ ID NO:1 or SEQ ID NO:2.

17-18. (canceled)

19. A replication composition for use in performance of PCR, comprising:
(a) a set of primer pairs selected from the group consisting of:
(i) one or more primer pairs comprising nucleic acid sequences comprising:
(A) SEQ ID NO:3 and SEQ ID NO:4;
(B) SEQ ID NO:28 and SEQ ID NO:29; and/or
(C) SEQ ID NO:30 and 31;
(ii) one or more primer pairs comprising nucleic acid sequences comprising:
(A) SEQ ID NO:7 and SEQ ID NO:8;
(B) SEQ ID NO:9 and SEQ ID NO:10;
(C) SEQ ID NO:11 and SEQ ID NO:12;
(D) SEQ ID NO:13 and SEQ ID NO:14; and/or
(E) SEQ ID NO:32 and SEQ ID NO:33; and
(iii) a combination thereof; and
(b) thermostable DNA polymerase.

20. The replication composition of claim 19 further comprising at least one probe/quencher pair selected from the group consisting of SEQ ID NO:5 and SEQ ID NO:6, SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, or SEQ ID NO:24 and SEQ ID NO:26, and a combination thereof.

21. A kit for detection of Salmonella enteritidis in a sample, comprising the replication composition of claim 20, wherein the kit comprises the primer pair of (a)(i) and the probe/quencher pair of SEQ ID NO:5 and SEQ ID NO:6.

22. A kit for detection of Salmonella typhimurium in a sample, comprising the replication composition of claim 20, wherein the kit comprises the one or more primer pairs of (a)(ii) and at least one probe/quencher pair selected from the group consisting of SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, or SEQ ID NO:24 and SEQ ID NO:26, and a combination thereof.

23. A kit for the detection of Salmonella enteritidis and/or Salmonella typhimurium in a sample, comprising the replication composition of claim 20, wherein the kit comprises the primer pair of (a)(i); the one or more primer pairs of (a)(ii); the probe/quencher pair of SEQ ID NO:5 and SEQ ID NO:6; and at least one probe/quencher pair selected from the group consisting of SEQ ID NO:15 and SEQ ID NO:16, SEQ ID NO:17 and SEQ ID NO:18, SEQ ID NO:19 and SEQ ID NO:20, SEQ ID NO:21 and SEQ ID NO:22, SEQ ID NO:21 and SEQ ID NO:23, SEQ ID NO:24 and SEQ ID NO:25, or SEQ ID NO:24 and SEQ ID NO:26, and a combination thereof.
